(19) United States
(12) Patent Application Publication
(10) Pub. No.: US 2011/0124718 A1
(43) Pub. Date: May 26, 2011

(54) Reduction Of Acetaminophen Toxicity By Dietary Milk Thistle Extract

(76) Inventor: Mark D. McCain, Wylie, TX (US)

(21) Appl. No.: 12/625,214

(22) Filed: Nov. 24, 2009

(51) Int. Cl. A61K 31/357; A61P 1/16

(52) U.S. Cl. 514/452

(57) ABSTRACT

The present invention is directed to the reduction of acetaminophen toxicity by dietary milk thistle extract. Milk thistle has been reported to have protective effects on the liver and to greatly improve its function. It is typically used to treat liver cirrhosis, chronic hepatitis (liver inflammation), and toxin-induced liver damage. The present invention, for the first time, combines milk thistle and acetaminophen in a unique and novel way to provide single dosages containing both ingredients, whether dispensed in a liquid suspension, chewable tablets, coated caplets, gelcaps, geltabs, and suppositories.

### CROSS-REFERENCE TO RELATED APPLICATIONS

None.

### TECHNICAL FIELD

This invention relates to the pain medication Acetaminophen in combination with dietary Milk Thistle extract.

### BACKGROUND

Acetaminophen belongs to a class of drugs called analgesics (pain relievers) and antipyretics (fever reducers). Acetaminophen relieves pain by elevating the pain threshold, that is, by requiring a greater amount of pain to develop before a person feels it. The FDA approved acetaminophen in 1951. Acetaminophen is often dispenses in a liquid suspension, chewable tablets, coated caplets, gelcaps, geltabs, and suppositories. Dosages are most often within the range of 325, 500 and 650 mg, with oral dosage for adults within the 325 to 650 mg range and the oral dose for a child within the range of 40-600 mg. Dosages are typically spread out over 4 to 6 hour periods. Acetaminophen is also used for the relief of various aches and pains associated with many conditions, such as arthritis or broken bones, etc.

Acetaminophen is metabolized by the liver. Therefore drugs that increase the action of liver enzymes that metabolize acetaminophen reduce the levels of acetaminophen and may decrease the action of acetaminophen. Doses of acetaminophen greater than the recommended doses are toxic to the liver and may result in severe liver damage. Although side effects with acetaminophen are few is used within the intended range, serious side effects such as liver damage can an do occur in large doses. Liver damage has also been seen in chronic use or use with alcohol or other drugs that can also damage the liver. The potential for acetaminophen to harm the liver is significantly raised when combined with alcohol or drugs.

Milk thistles are native to the Mediterranean regions of Europe, North Africa and the Middle East. The name “milk thistle” derives from two features of the leaves: they are mottled with splashes of white and they contain a milky sap. However, it is the seeds of milk thistle that herbalists have used for 2000 years to treat chronic liver disease and protect the liver against toxins. Milk thistle has been reported to have protective effects on the liver and to greatly improve its function. It is typically used to treat liver cirrhosis, chronic hepatitis (liver inflammation), and toxin-induced liver damage.

In clinical trials silymarin (the Milk Thistle extract) has typically been administered in amounts ranging from 50-500 mg per day in two to three divided doses. However higher doses have been studied, 600 or 1200 mg daily in patients chronically infected with hepatitis C virus. Prescriptions of 280 mg of a milk thistle standardized extract may be taken daily as a preventative. Milk Thistle provides hepatocelluler protection by stabilizing hepatic cell membranes. It alters the structure of the outer cell membrane of the hepatoytes in such a way as to prevent the penetration of the liver toxins into interior of the cell. When damage has already been done, “Milk Thistle” (silymarin) aids the liver in repairing injured cells and generating new ones. It does this by stimulating protein synthesis through the enzyme “RNA” polymerase. Milk Thistle (silymarin) prevents toxins from entering the liver by guarding the organ's numerous doorways—the membranes of liver cells. By slowing the rate at which the liver absorbs harmful substances, the toxins are excreted through the kidneys before they can cause liver damage.

The present invention, for the first time, combines milk thistle and acetaminophen in a unique and novel way to provide single dosages containing both ingredients, whether dispensed in a liquid suspension, chewable tablets, coated caplets, gelcaps, geltabs, and suppositories.

### SUMMARY

The purpose of the invention is to provide an improvement in acetaminophen therapy.

Another objective is to alleviate or prevent the undesirable side-effects of acetaminophen, such as liver damage or liver inflammation.

A further objective is to reduce high dose acetaminophen toxicity by dietary Milk Thistle (silymarin).

An additional object it to utilize Milk Thistle ingestion to provide a salutary benefit to those ingesting acetaminophen, by counter-acting and preventing the toxic affects of acetaminophen.

An objective is to provide a synergistic acetaminophen formulation comprising acetaminophen together with an effective amount of Milk Thistle

An objective' is to employ Milk Thistle to aid human patients suffering from diseases affecting the liver.

An objective is to medically use the combination of acetaminophen and Milk Thistle in a human subject, so that the acetaminophen may have pain relieving properties while reducing liver related toxicity.

An objective is to alleviate or prevent adverse side effects due to the chronic ingestion of acetaminophen.

### BRIEF DESCRIPTION OF THE INVENTION

None

### DETAILED DESCRIPTION

Traditional milk thistle extract is made from the seeds, which contain approximately 4-6% silymarin. The extract consists of about 65-80% silymarin (a flavonolignan complex) and 20-35% fatty acids, including linoleic acid. Silymarin is a complex mixture of polyphenolic molecules, including seven closely related flavonolignans (silybin A, silybin B, isosilybin A, isosilybin B, silychristin, isosilychristin, silydianin) and one flavonoid (taxifolin). Silibinin, a semipurified fraction of silymarin, is primarily a mixture of 2 diasteroisomers, silybin A and silybin B, in a roughly 1:1 ratio. In clinical trials silymarin has typically been administered in amounts ranging from 420-480 mg per day in two to three divided doses. However higher doses have been studied, such as 600 mg daily in the treatment of type II diabetes and 600 or 1200 mg daily in patients chronically infected with hepatitis C virus. An optimal dosage for milk thistle preparations has not been established; however, it has been found to be beneficial to reducing liver toxicity. See Lawrence, Valerie MD, MSc et al. (2000). “Milk Thistle: Effects on Liver Disease and Cirrhosis and Clinical Adverse Effects”. AHRQ Publication No. 01-E025. 420 mg (standardized 70-80 percent silymarin), taken daily for up to 12 weeks and up to 280 mg of a milk thistle standardized extract may be taken daily as a preventative.

What is claimed is:

1. A method for reducing toxicity and inhibiting liver damage of a mammal due to the ingestion of acetaminophen, comprising the concomitant ingestion of an amount of milk thistle extract or a precursor of milk thistle extract effective to reduce toxicity and inhibit ulcerogenesis and acetaminophen toxicity of a mammal due to the ingestion of acetaminophen, the amount being such as to provide a daily dosage greater than 50 milligrams but less than a toxic amount of milk thistle extract or milk thistle extract precursor.

2. The method of claim 1 in which the effective amount of milk thistle extract or a precursor of milk thistle extract is such as to provide a daily dosage in the range of 50 to 500 milligrams.

3. The method of claim 1 in which the effective amount of milk thistle extract or a precursor of milk thistle extract is such as to provide a daily dosage in the range of 501 to 1200 milligrams.

4. An ingestible composition which does not exhibit the adverse side effects caused by the ingestion of acetaminophen in mammals, comprising acetaminophen and an amount of milk thistle extract or milk thistle extract precursor effective to reduce and inhibit ulcerogenesis and liver toxicity of a mammal due to the ingestion of acetaminophen, in which the amount of milk thistle extract or milk thistle extract precursor is greater than 1 I.U. per milligram of acetaminophen but less than a toxic amount of milk thistle extract or milk thistle extract precursor.

5. The method of claim 1 in which said mammal is a human.

6. The composition of claim 4 in which said mammal is a human.

7. The composition of claim 4 in which the amount of milk thistle extract or milk thistle extract precursor per milligram of acetaminophen is from 1 to 50,000 I.U.

8. The method of claim 1 or the composition of claim 4 in which said milk thistle extract comprises silybin A, silybin B, isosilybin A, isosilybin B, silychristin, isosilychristin, silydianin, flavonoid (taxifolin), or other effective silybin.
